(19) United States
(12) Patent Application Publication
(10) Pub. No.: US 2021/0275408 A1
(43) Pub. Date: Sep. 9, 2021

(54) STICK-TYPE SHEET MASK AND MANUFACTURING METHOD THEREFOR

(71) Applicant: Yun Hee JANG, Gwangju-si (KR)

(72) Inventor: Yun Hee JANG, Gwangju-si (KR)

(21) Appl. No.: 17/270,585

(22) PCT Filed: Oct. 31, 2018
(86) PCT No.: PCT/KR2018/013083

§ 371 Date: Feb. 23, 2021

(30) Foreign Application Priority Data
Aug. 24, 2018 (KR) 10-2018-0099046

(51) Int. Cl. A61K 8/02; A61K 8/92; A61K 8/9789; A61K 8/73

(52) U.S. Cl. CPC A61K 8/0229; A61K 8/922; A61K 8/9789; A61K 2800/5922; A61K 2800/805; A61K 2800/262; A61K 8/735

(57) ABSTRACT

Provided is a stick-type mask pack according to an exemplary embodiment of the present invention, which is configured by mixing at least two natural vegetable oils and solid waxes and added with a liquid-crystalizing agent, a binder, and a clarifying agent to be transparent and maintain stability even at a high temperature, wherein a layer where a liquid crystal layer is in contact with the skin when applied to the skin forms a nutrient supply barrier that balances oil and moisture and a layer far away from the skin forms an air barrier.

### TECHNICAL FIELD

The present invention relates to a stick-type mask pack and a manufacturing method thereof, and more particularly, to a stick-type mask pack and a manufacturing method thereof, capable of supplying moisture to a dry skin anytime, anywhere, just by application without an existing stuffy mask sheet, and being applied freshly and softly to a rough skin in hot and dry environments without irritation.

### BACKGROUND ART

In general, a mask pack is attached to the face in a state impregnated with a cosmetic liquid containing various nutrients useful for the skin, such as a moisturizer and a whitening agent, to supply moisture and nutrition to the skin. In order to absorb a large amount of moisture and nutrition in the skin deeply, a method of attaching a mask pack made of non-woven fabric to the face for a certain period of time has been used.

In particular, in order to facilitate facial massage, various types of facial mask packs have been released so that non-woven fabric, cosmetic films, etc. are compressed into a certain form to be covered over the face to be massaged.

The massage liquid used in the mask pack selectively contains various ingredients depending on its purpose such as skin improvement and moisture supply, and recently, not only chemicals such as aromas, fragrances, and herbal medicines, but also natural ingredients capable of minimizing skin troubles have been used in trend.

These mask packs are usually used in a lying or sitting posture because the mask packs may fall off from the face if the mask packs are used in a standing position or moved.

In addition, since these mask packs are manufactured by impregnating the cosmetic liquid into a base sheet, nutrients contained in the cosmetic liquid are not concentrated on a skin contact surface but widely dispersed, which is not efficient, and in many cases, it is common that the nutrients flow down so as not to be used over again and are mostly discarded.

In order to solve this problem, when the mask packs are used for a long time, the sheet is dried and the skin is tightened or the air permeability decreases, and thus, rather, there is a problem to cause skin troubles.

In addition, these mask packs have a problem in that harmful ingredients such as preservatives are not only used for storage and maintenance, but also need to be washed or wiped separately after using the mask packs.

### PRIOR ARTS

(Patent Document 0001) 1. Korean Patent Registration No. 10-0782577 (Nov. 29, 2007)

(Patent Document 0002) 2. Korean Patent Registration No. 10-1650302 (Aug. 17, 2016)

(Patent Document 3) 3. Korean Patent Publication No. 10-2011-0078384 (Jul. 7, 2011)

(Patent Document 4) 4. Korean Utility Model Registration No. 20-0427765 (Sep. 25, 2006)

### DISCLOSURE

### Technical Problem

The present invention has been made in an effort to provide a stick-type mask pack and a manufacturing method thereof having advantages of efficiently supplying nutrients without waste by applying and using a solid ingredient with a high natural content in a simple use without a sheet.

Further, the present invention has been made in an effort to provide a stick-type mask pack and a manufacturing method thereof having advantages of being skin-friendly, having good moisturizing ability, having good permeability without a sheet while not using harmful ingredients such as preservatives, having no stickiness, being absorbed into the skin after 15 minutes to maintain moisture, and having no need to be washed or wiped separately.

### Technical Solution

An exemplary embodiment of the present invention provides a stick-type mask pack, which is configured by mixing at least two natural vegetable oils and solid waxes and added with a liquid-crystalizing agent, a binder, and a clarifying agent to be transparent and maintain stability even at a high temperature, wherein a layer where a liquid crystal layer is in contact with the skin when applied to the skin forms a nutrient supply barrier that balances oil and moisture and a layer far away from the skin forms an air barrier.

The wax may be formed of at least one of black elderberry wax, eclipta prostrata wax, rhus succedanea fruit wax, acacia decurrens flower wax, and jojoba ultrafine powder.

The at least two or more natural vegetable oils may be formed with at least one of 2-octyldodecanol canola oil, argania spinosa kernel oil, avocado oil, grapeseed oil, camellia oil, sunflower seed oil, macadamia ternifolia seed oil, olive oil, rosemary leaf oil, lavender oil, evening primrose oil, apricot seed oil, leptospermum petersonii oil, adansonia digitata seed oil, babassu seed oil, moringa oleifera seed oil, euterpe oleracea fruit oil, eucalyptus leaf oil, jojoba seed oil, and essential oil.

The diisostearylmaleate may be used as the clarifying agent, 2-octyldodecanol may be used as the liquid-crystallizing agent, and dibutyl lauroyl glutamide and dibutyl ethylhexanoylgulutamide may be used as the binder.

The stick-type mask pack may further include hyaluronic acid and beta-glucan in order to form the nutrient supply barrier, wherein the beta-glucan may be extracted from sparassis crispa.

Another exemplary embodiment of the present invention provides a manufacturing method of a stick-type mask pack comprising the steps of: boiling a wax solid to 130° C., cooling a liquid wax boiled to 130° C. to 100° C.; mixing at least two or more natural oils with the liquid wax cooled to 100° C.; stirring the mixture of the liquid wax and the natural oils by adding a liquid-crystallizing agent, a binder, and a clarifying agent; and pouring and solidifying the mixture into a mold after the stirring.

The wax solid, the natural oils, the liquid-crystallizing agent, the binder, and the clarifying agent may all have a stability grade of 1 to 2 for each cosmetic ingredient.

The wax solid may be formed as a liquid crystal formulation by using black elderberry wax, eclipta prostrata wax, rhus succedanea fruit wax, acacia decurrens flower wax, and jojoba wax ultrafine powder.

According to the stick-type mask pack of the exemplary embodiment of the present invention, it is possible to efficiently supply nutrients without waste by applying and using a solid ingredient with a high natural content in a simple use without a sheet.

Further, the stick-type mask pack of the exemplary embodiment of the present invention is skin-friendly, has good moisturizing ability, has good permeability without a sheet while not using harmful ingredients such as preservatives, has no stickiness, has a nutrient barrier and an air barrier formed when a liquid crystal layer is in contact with the skin to be molten by a skin temperature and absorbed into the skin after 15 minutes to maintain moisture, and has no need to be washed or wiped separately.

### DESCRIPTION OF THE DRAWINGS

FIG. 1 is a flowchart illustrating a manufacturing method of a stick-type mask pack according to an exemplary embodiment of the present invention.

FIG. 2 is a photograph illustrating a configuration of a stick-type mask pack according to an exemplary embodiment of the present invention.

FIG. 3 is a schematic diagram illustrating a using method of a stick-type mask pack according to an exemplary embodiment of the present invention.

FIG. 4 is a cross-sectional view for describing technical features of a stick-type mask pack according to an exemplary embodiment of the present invention.

### MODE FOR INVENTION

In the following detailed description, only certain exemplary embodiments of the present invention have been shown and described, simply by way of illustration. As those skilled in the art would realize, the described embodiments may be modified in various different ways, all without departing from the spirit or scope of the present invention. Accordingly, the drawings and description are to be regarded as illustrative in nature and not restrictive. Like reference numerals designate like elements throughout the specification.

In addition, unless explicitly described to the contrary, the word “comprise”, and variations such as “comprises” or “comprising”, will be understood to imply the inclusion of stated elements but not the exclusion of any other elements. In addition, the terms “-er”, “-or” and “module” described in the specification mean units for processing at least one function and operation and may be implemented by hardware components or software components and combinations thereof.

Hereinafter, a stick-type mask pack and a manufacturing method thereof according to an exemplary embodiment of the present invention will be described in detail with reference to the accompanying drawings.

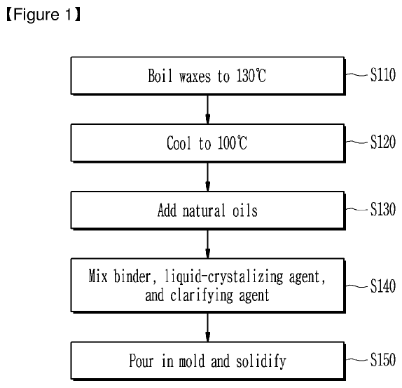

As illustrated in FIG. 1, in a manufacturing method of a stick-type mask pack according to an exemplary embodiment of the present invention, first, a wax solid is put in a container and boiled while stirring to 130° C. (S110).

After a burner is turned off, a liquid wax boiled to 130° C. is cooled to 100° C. (S120).

The liquid wax cooled to 100° C. is mixed with at least two natural oils (S130).

A liquid-crystallizing agent, a binder, and a clarifying agent are added to the mixture of the liquid wax and the natural oils, stirred (S140), and then poured into a mold to be solidified (S150). [symbol] [symbol] [symbol] [symbol] [symbol] [symbol] (S140) [symbol] [symbol] (S150).

The wax solid, the natural oils, the liquid-crystallizing agent, the binder, and the clarifying agent are all characterized in that the stability grade for each cosmetic ingredient is 1 to 2. Since a ratio of each ingredient is made to be almost 1:1, purified water or the like is not separately added, so that there is no need to add a preservative, etc. required in the case of a sheet substrate.

The at least two or more natural vegetable oils are formed with at least one of 2-octyldodecanol canola oil, argania spinosa kernel oil, avocado oil, grapeseed oil, camellia oil, sunflower seed oil, macadamia ternifolia seed oil, olive oil, rosemary leaf oil, lavender oil, evening primrose oil, apricot seed oil, leptospermum petersonii oil, adansonia digitata seed oil, babassu seed oil, moringa oleifera seed oil, euterpe oleracea fruit oil, eucalyptus leaf oil, jojoba seed oil, and essential oil, and have a ratio with other ingredients of 1:1 even when these oils are mixed and used.

Diisostearylmaleate may be used as the clarifying agent, 2-octyldodecanol may be used as the liquid-crystallizing agent, and dibutyl lauroyl glutamide and dibutyl ethylhexanoylgulutamide may be used as the binder.

It may be seen that the clarifying agent, the liquid crystallizing agent, and the binder liquid-crystalize black elderberry wax, eclipta prostrata wax, baobab seed oil, babassu seed oil, and moringa oleifera seed oil, so as to be transparent and stable even at a high temperature.

Herein, the liquid crystallization means an intermediate state between a liquid and a crystal. Wax and natural oils are configured to have molecules which are arranged in an irregular liquid state in one direction by the liquid-crystallizing agent, but arranged in a regular crystal state in the other direction. As described in detail below, it means that the regular molecular state becomes an irregular liquid state in an area where the skin temperature reaches 36.5° C.

Meanwhile, it may be seen that the black elderberry wax strengthens immunity and has a great natural antioxidant effect, and contains anthocyanin/carolene/tannin/vitamin c/vitamin b/vitamin a/amino acid as main ingredients.

In addition, it may be seen that the eclipta prostrata wax has a skin vitality effect and improves skin diseases, and contains chaponin, tannin, ecryptin, coumarin, wetractin as a compound, and vitamin A as main ingredients.

The baobab seed oil serves to alleviate and protect skin diseases, and is rich in vitamin c, calcium, and iron as well as citric acid and tartaric acid.

The babassu seed oil has a melting point similar to the skin temperature to have rapid absorption, forms a natural coating film on the skin to help in forming a protective film, is rich in vitamin e, and the moringa oleifera seed oil plays a role in reorganizing the skin barriers.

On the other hand, in an exemplary embodiment of the present invention, instead of solid waxes, the wax is formed of at least one of black elderberry wax, eclipta prostrata wax, rhus succedanea fruit wax, acacia decurrens flower wax, and jojoba ultrafine powder. The ultrafine powder may be preferably easily used as a mask pack by performing liquid crystallization in which molecular crystals are arranged side by side in one direction when the liquid-crystallizing agent is added.

For the stick-type mask pack prepared as described above, stability, transparency, absorption time, dryness, and skin irritation were compared and evaluated, respectively.

### EXPERIMENTAL EXAMPLE 1

### Evaluation of Transparency and Stability

As shown in Table 1, with respect to 100 stick-type mask pack samples under different temperature conditions, phase separation of oil and wax, and smearing and suspension of wax were observed with the naked eye for 100 days, and then the results were shown.

TABLE 1

| | −10° C. | 0° C. | 20° C. | 30° C. | 40° C. |
|---|---|---|---|---|---|
| 20 day | Good | Good | Good | Good | Good |
| 40 day | Good | Good | Good | Good | Good |
| 70 day | Good | Good | Good | Good | Good |
| 100 day | Good | Good | Good | Good | Good |

### EXPERIMENTAL EXAMPLE 2

### Evaluation of Absorption Time of Nutrients, Dryness, Permeability, and Skin Irritation

As shown in Table 2, 3 g of stick-type mask pack samples were applied to the faces of 30 subjects in their 30s to 40s five times at intervals of 2 days, and then the time to be completely absorbed was measured. After completely absorbing the nutrients, sensory evaluation was conducted on dryness, permeability, skin irritation and whether the makeup was applied well, and the results were shown by averaging obtained scores. Here, the higher the dryness, permeability, skin irritation and whether the makeup was applied well, the closer to 10 points were made.

Whether the makeup was applied well was tested by using one type of same cushion, such as soft foundation.

TABLE 2

| | | Two | Three | Four | Five |
|---|---|---|---|---|---|
| | Once | times | times | times | times |
| Absorption | 10 | 12 | 14 | 13 | 14 |
| time | minutes | minutes | minutes | minutes | minutes |
| Dryness | 2.6 | 3.7 | 2.7 | 3.2 | 3 |
| Permeability | 7.2 | 7.3 | 7.7 | 7.6 | 7.7 |
| and whether | | | | | |
| makeup is | | | | | |
| applied well | | | | | |
| thereafter | | | | | |
| Skin | 3 | 3.1 | 2.8 | 3.1 | 3 |
| irritation | | | | | |

As may be seen from Table 2, when the absorption time of nutrients was about 10 to 15 minutes, most of the subjects felt that the nutrients were absorbed, and after using the stick-type mask pack, the dryness was almost not felt, permeability was good, and little skin irritation was felt.

Therefore, it could be seen that the stick-type mask pack according to the exemplary embodiment of the present invention was skin-friendly with natural oil ingredients compared to existing sheet packs, excellent in moisturizing by using at least two essential oils, and an oil moisturizing film had a regeneration effect, skin elasticity, gloss, and skin surface cleanup.

In addition, in the manufacturing method of the stick-type mask pack according to the exemplary embodiment of the present invention, since the stick-type mask pack was manufactured by solidifying natural oils, it could be seen that preservatives, etc. that were widely used in existing sheet mask packs and caused skin irritation, may not be used, and permeability was good without a sheet, so that the makeup was applied well when the stick-type mask pack was used before makeup.

In addition, the stick-type mask pack according to the exemplary embodiment of the present invention has no stickiness, and may be absorbed into the skin after 15 minutes to maintain moisture, and does not need to be washed or wiped separately, and maintains a moist skin texture and prevents dryness when used continuously, and may feel the excellence compared to existing sheet mask packs, mists, etc.

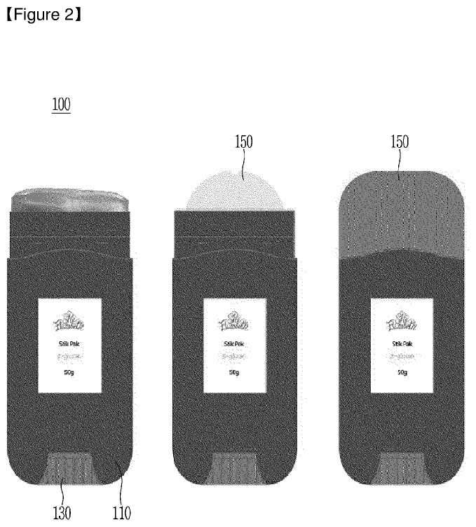

As illustrated in FIG. 2, the stick-type mask pack 100 according to the exemplary embodiment of the present invention manufactured as described above is configured to be filled in the stick container 110, adjusted in height by a screw type height adjustment device 130 installed at the bottom of the stick container 110, and then taken out to about 5 mm and applied.

Meanwhile, in order to prevent contamination before and after use and to maintain airtightness, an inner protective cap 150 may be further provided, and an outer cap 170 may be further provided outside.

The inner protective cap 150 is made of a silicone material and blocks the contact with air to prevent the oil ingredients from being rancid.

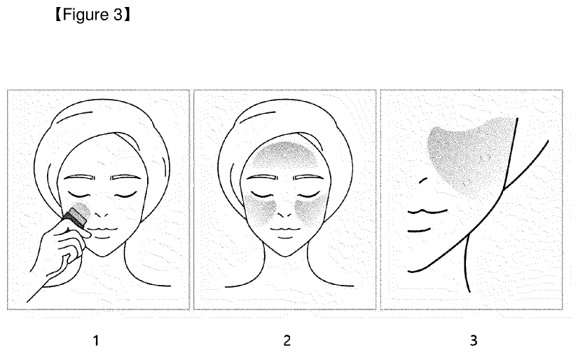

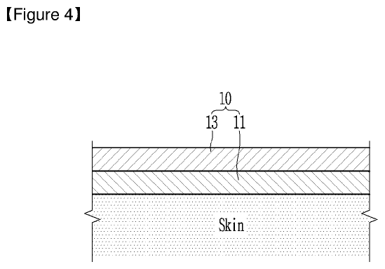

As illustrated in FIGS. 3 and 4, the stick-type mask pack 100 according to the exemplary embodiment of the present invention is configured by mixing at least two natural vegetable oils and solid waxes and added with the liquid-crystalizing agent, the binder, and the clarifying agent to form the liquid crystal layer 10 that is transparent and maintains stability even at a high temperature. As illustrated in FIG. 3, when applied to the skin, a layer where the liquid crystal layer 10 is in contact with the skin forms the nutrition supply barrier 11 that balances oil and moisture to supply nutrition, and a layer away from the skin may form a lot of air barriers by an effect of abundant unsaturated fatty acids to form the air barrier 13 that is not stuffy or greasy.

That is, it is possible to balance the oil and moisture of a dry skin and maintain moisture in the skin by the nutrient supply barrier, and to support and maintain nutrients so that nutrition is sufficiently supplied without feeling stuffy or oily by the air barrier.

Therefore, the stick-type mask pack may be applied freshly and gently to a rough skin in a hot and dry environment without irritation, and may supply moisture to a dry skin anytime, anywhere just by applying without the existing stuffy mask sheet.

It may be seen that when a complex consisting of 20 kinds of natural vegetable oils and all ingredients have 100% of ewg(0-1) green grade which is a cosmetic stability grade, there is provided a liquid crystal formulation which is transparent, stable, safe in the skin, and permeable.

Accordingly, as illustrated in FIG. 3, when the outer and inner caps 150 and 170 of the stick-type mask pack 100 are opened and the height adjustment device 130 is turned to apply the stick-type mask pack to the face, the liquid crystal layer 10 reacts and is molten at the skin temperature to form the nutrient supply barrier 11. In addition, while the air barrier 13 supports nutrients so that nutrition is sufficiently supplied, the air barrier 13 forms an air film to maintain permeability, so that the stick-type mask pack 100 may not be felt stuffy even while applying the stick-type mask pack 100.

The nutrients contain beta-glucan present in yeast cell walls, mushrooms, grains, etc., and particularly, the beta-glucan is extracted from sparassis crispa to have a better skin moisturizing function than hyaluronic acid and form a protective film, strengthen the skin barrier, protect the skin from external irritation, promote the production of growth factors in epithelial cells, play a role of skin antioxidation as natural high molecular polysaccharides, and increase a skin soothing effect.

<Description of symbols>

| | | | |
|---|---|---|---|
| 10: | Liquid crystal layer | 11: | Nutrient supply barrier |
| 13: | Air barrier | 110: | Container body |
| 130: | Height adjustment device | 150: | Inner cap |
| 170: | Outer cap | | |

1. A stick-type mask pack which is configured by mixing at least two natural vegetable oils and solid waxes and added with a liquid-crystalizing agent, a binder, and a clarifying agent to be transparent and maintain stability even at a high temperature, wherein a layer where a liquid crystal layer is in contact with the skin when applied to the skin forms a nutrient supply barrier that balances oil and moisture and a layer far away from the skin forms an air barrier.

2. The stick-type mask pack of claim 1, wherein
the wax is formed of at least one of black elderberry wax, eclipta prostrata wax, rhus succedanea fruit wax, acacia decurrens flower wax, and jojoba ultrafine powder.

3. The stick-type mask pack of claim 1, wherein
the at least two or more natural vegetable oils are formed with at least one of 2-octyldodecanol canola oil, argania spinosa kernel oil, avocado oil, grapeseed oil, camellia oil, sunflower seed oil, macadamia ternifolia seed oil, olive oil, rosemary leaf oil, lavender oil, evening primrose oil, apricot seed oil, leptospermum petersonii oil, adansonia digitata seed oil, babassu seed oil, moringa oleifera seed oil, euterpe oleracea fruit oil, eucalyptus leaf oil, jojoba seed oil, and essential oil.

4. The stick-type mask pack of claim 1, wherein
diisostearylmaleate is used as the clarifying agent, 2-octyldodecanol is used as the liquid-crystallizing agent, and dibutyl lauroyl glutamide and dibutyl ethylhexanoylgulutamide are used as the binder.

5. The stick-type mask pack of claim 1, further comprising:
hyaluronic acid and beta-glucan in order to form the nutrient supply barrier, wherein the beta-glucan is extracted from sparassis crispa.

6. A manufacturing method of a stick-type mask pack comprising the steps of:
boiling a wax solid to 130° C.;
cooling a liquid wax boiled to 130° C. to 100° C.;
mixing at least two or more natural oils with the liquid wax cooled to 100° C.; stirring the mixture of the liquid wax and the natural oils by adding a liquid-crystallizing agent, a binder, and a clarifying agent; and
pouring and solidifying the mixture into a mold after the stirring.

7. The manufacturing method of the stick-type mask pack of claim 6, wherein
the wax solid, the natural oils, the liquid-crystallizing agent, the binder, and the clarifying agent all have a stability grade of 1 to 2 for each cosmetic ingredient.

8. The manufacturing method of the stick-type mask pack of claim 6, wherein
the wax solid is formed as a liquid crystal formulation by using black elderberry wax, eclipta prostrata wax, rhus succedanea fruit wax, acacia decurrens flower wax, and jojoba wax ultrafine powder.
